(19) United States
(12) Patent Application Publication
(10) Pub. No.: US 2026/0269027 A1
(43) Pub. Date: Sep. 10, 2026

(54) METHOD OF ANALYZING TUMOR, SYSTEM FOR ANALYZING TUMOR, AND METHOD OF GENERATING ANALYZED DATA OF TUMOR

(71) Applicant: Sony Group Corporation, Tokyo (JP)

(72) Inventors: Hirokazu Tatsuta, Kanagawa (JP); Kazuhiro Nakagawa, Saitama (JP); Tomohiko Nakamura, Tokyo (JP); Masahiro Matsumoto, Kanagawa (JP); Seiji Wada, Kanagawa (JP); Yoshihito Hayashi, Chiba (JP)

(73) Assignee: Sony Group Corporation, Tokyo (JP)

(21) Appl. No.: 19/166,275

(22) PCT Filed: Mar. 13, 2024
(86) PCT No.: PCT/JP2024/009832

§ 371 Date: Sep. 17, 2025

(30) Foreign Application Priority Data
Mar. 31, 2023 (JP) 2023-058887

(51) Int. Cl. G16H 10/40; G16H 50/20

(52) U.S. Cl. CPC G16H 10/40; G16H 50/20

(57) ABSTRACT

To provide a method of analyzing a tumor, a system for analyzing a tumor, and a method of generating analyzed data of a tumor that are effective and of high quality.

According to the present technology, there is provided a method of analyzing a tumor to acquire information regarding a tumor by using an examination of a tumor tissue and chronological examinations of blood. According to the present technology, there is also provided a method of generating analyzed data of a tumor by using an examination of a tumor tissue and chronological examinations of blood and having the examinations intervene in each other to integrally analyze data from the examinations, thereby generating data regarding the tumor.

### TECHNICAL FIELD

The present technology relates to a method of analyzing a tumor, a system for analyzing a tumor, and a method of generating analyzed data of a tumor. More specifically, the present technology is concerned with a method of analyzing a tumor, a system for analyzing a tumor, and a method of generating analyzed data of a tumor that are effective and of high quality.

### BACKGROUND ART

According to Table 13 and Table 14 of “National Medical Care Expenditure, 2019” published by Ministry of Health, Labor, and Welfare of Japan, in terms of the rates of medical expenses spent for diseases, the highest percentage is 13% for malignant neoplasms (malignant tumors), topping cerebrovascular diseases (5.7%), hypertensive diseases (5.5%), and diabetes (3.8%). Moreover, according to Table 6 of “Overview of Vital Statistics (Corrected Figures), 2020” published by Ministry of Health, Labor, and Welfare of Japan, in terms of cause-specific desire rates, the highest percentage is 28% for malignant neoplasms, outdoing heart diseases (15%) and cerebrovascular diseases (8%). In view of these facts, efforts to make more efficient analyses regarding tumors including malignant tumors can be said to have a significant social impact.

Incidentally, PTL 1, for example, discloses a machine-learning-based clinical predicting method that predicts whether a test subject is resistive or sensitive to treatment with regard to a specific drug (platinum drug) against an ovarian cancer or predicts the survival rate of a subject having a lung cancer that responds to an immunotherapy. According to PTL 1, a survival rate and a hazard ratio are learned from the extraction of features from a tissue image and a subset of molecular information of a subject, and clinical prediction is made.

In addition, PTL 2 discloses a method of constructing an ex-vivo lung cancer detecting mathematical model based on a multiomics analysis that has increased the sensitivity and specificity of a diagnosis by detecting lung cancer markers in combination. According to PTL 2, it is proposed to use a logistic regression analysis as a process of integrating blood data and tissue data.

### CITATION LIST

### Patent Literature

### [PTL 1]

U.S. Patent Application Publication No. 2020/0105413

### [PTL 2]

PCT Patent Publication No. WO2021/238086

### SUMMARY

### Technical Problem

However, the conventional art has not sufficed yet to provide an analysis that is effective and of high quality with regard to tumors. Tumor, though it sounds simple itself, is highly diverse in meaning depending on organs, individuals, stages, treatment statuses, and drugs used, for example. Generally, the efficacy of a drug used on a tumor is reduced over a certain period of time, and the drug needs to be changed to another drug because of a transition of the diversity of the tumor. Hence, for predicting the transition, it is not enough to only observe the state of the tumor at a certain point of time, and it is necessary to observe or predict the state of the tumor as it transitions from time to time.

By contrast, in general, a tissue biopsy makes it possible to perform direct and various analyses as it analyzes a tumor tissue itself. However, since the tissue biopsy is highly invasive, the frequency at which to acquire tissue biopsy information has to be low due to a burden that the tissue biopsy poses on the subject or the like, and the tissue biopsy information only represents the state of the tumor at one time. Meanwhile, a liquid biopsy is able to acquire chronological data of a tumor and can provide an index representing the present state of the tumor owing to the short serum half-lives of circulating tumor cells, circular tumor DNAs, and the like. However, the liquid biopsy is unable to obtain as diverse kinds of data as tumor tissues, and pieces of liquid biopsy data themselves are often fragmentary.

Therefore, it is an object to the present technology to provide a method of analyzing a tumor, a system for analyzing a tumor, and a method of generating analyzed data of a tumor that are effective and of high quality.

### Solution to Problem

According to the present technology, there is provided a method of analyzing a tumor to acquire information regarding a tumor by using an examination of a tumor tissue and chronological examinations of blood.

According to the present technology, there is also provided a system for analyzing a tumor, including an information processing apparatus that includes a controller including an information acquiring section for acquiring information regarding an examination of a tumor tissue and chronological examinations of blood, an integrating section for integrating the information from the information acquiring section to generate data regarding the tumor, and an outputting section for outputting the data generated by the integrating section, a storing section for storing the data output from the outputting section, a displaying section for displaying the data output from the outputting section, and a communicating section for presenting the data output from the outputting section onto a network, a tumor tissue examination result outputting apparatus for outputting a result of the examination of the tumor tissue, and a blood examination result outputting apparatus for outputting a result of the examinations of the blood.

According to the present technology, there is further provided a method of generating analyzed data of a tumor by using an examination of a tumor tissue and chronological examinations of blood and having the examinations intervene in each other to integrally analyze data from the examinations, thereby generating data regarding the tumor.

Note that, in the present description, “biopsy” means an examination using a sample taken from a living body. An examination of a tumor tissue may be referred to as “tissue biopsy,” and an examination of blood as “liquid biopsy.” Moreover, “tumor” refers to abnormally grown cells and means cells that continue to grow even after the cause of the abnormal growth disappears or is removed. Specifically, the tumor represents a concept that covers benign tumors and malignant tumors. According to the present technology, in particular, the tumor refers to a malignant tumor (cancer). Further, “subject” means a human to be examined and represents a concept that covers a healthy individual, a person suspected of being affected with a benign tumor or a malignant tumor, a person affected with a benign tumor or a malignant tumor, and the like. According to the present technology, in particular, the subject refers to a person affected with a malignant tumor.

In the present description, further, “tumor tissue” represents a concept that covers a tumor cell (including a cell and an intercellular structure (for example, cytoplasm, cell membrane, nucleus, or the like)), a tumor region, a non-tumor region, a connective tissue, a blood vessel, a blood vessel wall, a lymphatic vessel, a fibrotic structure, a necrotic region, an organ, an organ fragment, or the like of a human and an animal. Moreover, “blood” represents a concept that covers blood, blood with some components removed therefrom by a centrifuge, for example, and blood to which any of various drugs and/or various reagents have been added, of a human and an animal.

### BRIEF DESCRIPTION OF DRAWINGS

FIG. 1 is a schematic conceptual diagram illustrating a method of analyzing a tumor according to an embodiment.

FIG. 2 is a diagram illustrating an example of a concept according to an analysis example 1.

FIG. 3 is a flowchart illustrating a flow example 1 of the analysis example 1.

FIG. 4 is a flowchart illustrating a flow example 2 of the analysis example 1.

FIG. 5 is a flowchart illustrating a flow example 3 of the analysis example 1.

FIG. 6 is a diagram illustrating an example of a concept according to an analysis example 2.

FIG. 7 is a flowchart illustrating a flow example of the analysis example 2.

FIG. 8 is a diagram illustrating an example of biomarkers obtained from a subject by an examination of a tumor tissue and an example of standards for administering drugs in an analysis example 3.

FIG. 9 is a diagram illustrating an example of a concept of application of the analysis example 3.

FIG. 10 is a diagram illustrating an example of a concept according to an analysis example 4.

FIG. 11 is a diagram illustrating an example of a concept according to an analysis example 5.

FIG. 12 is a block diagram illustrating an example of a system for analyzing a tumor.

FIG. 13 is a schematic conceptual diagram illustrating a modification of a database.

FIG. 14 is a block diagram illustrating an example of a hardware configuration of an information processing apparatus.

### DESCRIPTION OF EMBODIMENTS

Preferred embodiments for reducing the present technology to practice will be described hereinbelow with reference to the drawings.

The embodiments to be described below are illustrative of examples of representative embodiments of the present technology and should not make the scope of the present technology unduly narrow in interpretation. Note that details of the embodiments will be described below in the following order:
1. First embodiment (a method of analyzing a tumor)
(1) Background of a method of analyzing a tumor according to the present embodiment
(2) Analysis example 1
(2-1) Cocktail generator
(2-2) ctDNA analysis
(2-3) Prediction generator
(2-4) Others
(3) Flow examples of analysis example 1
(3-1) Flow example 1
(3-2) Flow example 2
(3-3) Flow example 3
(4) Analysis example 2
(5) Analysis example 3
(6) Analysis example 4
(7) Analysis example 5
2. Second embodiment (a system 1 for analyzing a tumor)
(1) Information acquiring section 11
(2) Integrating section 12
(3) Outputting section 13
(4) Storing section 14
(5) Displaying section 15
(6) Communicating section 16
(7) Tumor tissue examination result outputting apparatus 6
(8) Blood examination result outputting apparatus 7
(9) Database 2
(10) Example of a hardware configuration of an information processing apparatus 3
3. Third embodiment (a method of generating analyzed data of a tumor)

### 1. First Embodiment (a Method of Analyzing a Tumor)

A method of analyzing a tumor according to the present embodiment acquires information regarding the tumor by using an examination of a tumor tissue and chronological examinations of blood. In other words, the method integrally analyzes information obtained from a tissue biopsy and information obtained from a liquid biopsy, thereby making the biopsies complement each other's weaknesses and providing an analysis result that is more effective and highly reliable. Specifically, in reference to molecular information including genome, epigenome, and transcriptome, for example, obtained from the tissue biopsy and the chronological information concerning a blood analysis, feedback is given to a treatment policy and an examination policy. As a result, it is possible to provide a medical care that is effective and of high quality and to, in some cases, reduce unwanted treatments and minimize the physical burden on the subject and the treatment cost.

The method of analyzing a tumor according to the present embodiment will be described in detail below.

### (1) Background of a Method of Analyzing a Tumor According to the Present Embodiment

According to an examination of a tumor tissue, it is possible to acquire a tissue of a tumor or a normal tissue around the tumor, and diverse analytic processes are available to acquire detailed information of the tumor. So far, various analytic processes have been proposed for analyzing tumor tissue samples, and new analytic processes are being proposed.

Analytic processes that are in actual use at present are set forth below. Specifically, they include, for example, a morphological analysis based on HE hematoxylin and eosin staining, a marker analysis based on immunostaining owing to IHC, a genome transcriptome analysis where a tumor tissue sample or a tumor region is extracted by laser microdissection, and the like. Moreover, in research fields, analytic processes for a tumor microanalysis have been proposed. Specifically, for example, they include a spatial analysis for a tumor and immune cells around the tumor that uses multiplex fluorescent staining, a single-cell analysis for separating a single cell from a tumor tissue and analyzing the single cell for genome or transcriptome, and a spatial genomics analysis that is a single-cell analysis linked with spatial information.

However, processes of acquiring a tumor tissue range from a process called a needle biopsy to a surgical tumor removal process, for example. These processes are basically highly invasive to the examinee, and their frequency at which to acquire tumor information is extremely limited in exchange for the amount of information that can be obtained.

Meanwhile, a blood examination is an analytic process for analyzing circulating tumor cells (hereinafter also referred to as “CTCs”) in blood, cell free DNAs and circular tumor DNAs (hereinafter also referred to respectively as “cfDNAs” and “ctDNAs”) in plasma, and extracellular vesicles (hereinafter also referred to as “EVs”), using 4 to 10 mL of blood, for example (see M. Nikanjam, Liquid biopsy: current technology and clinical applications, Journal of Hematilogy & Oncology, 15, 131 (2022)).

Since a blood examination uses a sample of blood, the process of acquiring the sample is minimally invasive to the examinee and the blood examination is able to obtain chronological information relatively simply, compared to the process of acquiring samples and information of tumor tissues. For example, although an analytic process for targeting CTCs suffers a limited number of tumor cells contained in a blood sample, it can perform various analyses including an analysis of cells themselves, an analysis of genetic mutations, an expression analysis based on RNA, and a protein analysis.

However, there are no markers available for all tumor cells, making cell fractionation itself challenging. cfDNAs are present in the plasma of healthy persons (approximately 10 ng/ml), and it is known that subjects affected with malignant tumors tend to have increased variations of the number of cfDNAs and their fragment lengths. Consequently, a genome analysis and a methylation analysis, for example, of tumors can be performed using cfDNAs. Moreover, ctDNAs are highly likely to be present as nucleosome units in blood, and their fragments are present as 160 to 200 bases and their multiples. Furthermore, inasmuch as CTCs, cfDNAS, and ctDNAs are decomposed in the human body (liver and kidneys), their half-life is short, ranging from 16 to 114 minutes (see Liquid Biopsy; Fundamentals and Outlook of ctDNA http://archive.jsco.or.jp/data/jp/detail_images/56/ES-6.pdf).

From the foregoing, blood can be said to be an indicator of the state of an examinee at the time of taking a blood sample from him/her, unlike a tumor tissue that is formed of tumor cells accumulated over a certain period of time.

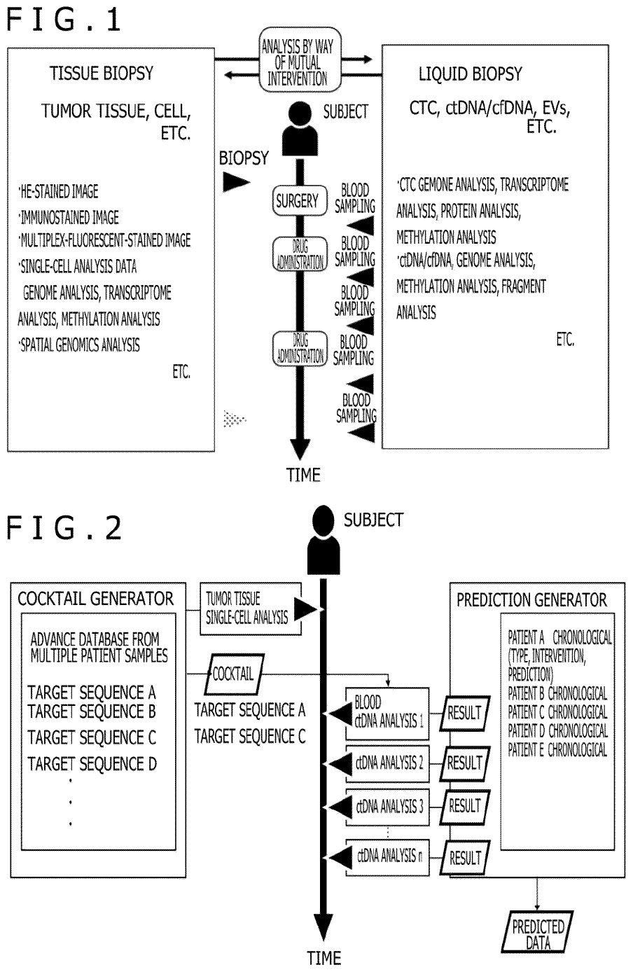

Consequently, according to the present embodiment, there is proposed a method of obtaining a more accurate analysis result by making tissue biopsy data and liquid biopsy data that have different characteristics complement each other. FIG. 1 is a schematic conceptual diagram illustrating a method of analyzing a tumor according to the present embodiment. As illustrated in FIG. 1, according to the present embodiment, the number of highly invasive examinations of tumor tissues conducted on a subject is reduced whereas the frequency at which minimally invasive blood examinations are conducted on the subject is set to a high value, with the results acquired as chronological data.

Here, according to the conventional art, much machine learning is performed on general multimodal data. By contrast, the present technology is novel in that, since it handles chronological data, it has an analysis flow for giving feedback from one set of analyzed data to another set of analyzed data, based on chronological phenomenon understanding. A process of handling chronological data or taking chronological data into account can increase prediction accuracy, and one analysis result and another treatment policy, examination policy, and analysis result affect each other, so that guidelines can be provided to give optimum treatments to respective subjects at appropriate timings.

Each of analysis examples in which examinations intervene in each other to integrally analyze data from the examinations and present an analysis result will be described below. Note that, though humans are to be examined in the analysis examples to be described below, the present embodiment is also applicable to animals when necessary.

### (2) Analysis Example 1

As described above, since mutations of tumor tissues are diverse, there are limitations on blood examinations and prognostic monitoring based on target markers extracted from a general-purpose data set. Hence, according to the present analysis example 1, a process of solving such a problem is proposed.

FIG. 2 is a diagram illustrating an example of a concept according to the analysis example 1. A single-cell genetic analysis is performed on tumor tissues of subjects affected with malignant tumors, the tumor tissues being acquired from an examination that has been conducted on the tumor tissues, and the state of genetic mutation is measured. Based on the measured data, a target cocktail of each of the subjects whose target genes have been selected for a blood examination is generated. The number of target cocktails is not limited to any value, and at least one target is determined by referring to the data regarding the examination of the tumor tissues. Then, a blood cfDNA of the subject is analyzed by a dPCR or a next-generation sequencer (hereinafter also referred to as an “NGS”) by using the generated target cocktail. In this fashion, there is established a target that is more individualized than target cocktails generated from data of many subjects (especially, subjects affected with malignant tumors), making it possible to perform more accurate prognostic monitoring.

### (2-1) Cocktail Generator

Operation of a cocktail generator illustrated in FIG. 2 will be described below.

Incidentally, the target cocktail may be an actual reagent or may be a data set for analyses rather than an actual reagent.

First, a genetic analysis of a tumor tissue part is performed. Specifically, a bulk analysis for analyzing an acquired tumor block in its entirety or a single-cell genomics analysis can be used, and an analysis using an NGS is assumed. Then, a sequence data set of genetic mutations of the tumor tissue from the subject is generated. Pieces of sequence data from the NGS contain all mutations in a coverage of certain reliability or larger.

Then, the pieces of sequence data are compared with a common mutation library obtained from samples that have been acquired in advance from multiple subjects (especially, subjects affected with malignant tumors) or an existing mutant gene library that is well known, establishing targets that can be cfDNA markers. Further, at this time, a target cocktail list is generated in a manner to distinguish common library components and sequences derived from the respective subjects from each other. Note that the degree to which they are distinguished may be adjusted to a desired state by appropriately changing weighting, setting the extent of weighting to a lower level, or setting the extent of weighting to a higher level.

### (2-2) ctDNA Analysis

A ctDNA analysis in blood examinations will be described below.

Based on the generated target list, a ctDNA having target mutations from a cfDNA derived from the subject is concentrated and NGS-analyzed. A probe group that hybridizes the established target mutations is generated or a probe cocktail is generated by being selected from prefabricated probes. If a probe cocktail that covers all assumed mutations can be prepared, then it is not necessary to generate a probe cocktail dedicated to the subject, and only a target mutation list can be used as an analysis target by an analysis in a subsequent stage. In addition, in the case where a few targets can be extracted, a dPCR rather than an NGS can be used.

Regarding the generation of a probe sequence, a target sequence and a probe sequence to be used for concentration are not necessarily required to be in complete agreement with each other, and a sequence including part of a probe sequence or a sequence associated with a human genome reference sequence (for example, an adjacent sequence) may be used. This is because a cfDNA has such characteristics that its part coiled around the histone is liable to remain and its region where chromatin is open (transfer region) is less liable to remain. When pieces of NGS data are analyzed, the analysis is performed in a manner to distinguish a sequence derived from the subject and a sequence selected from the library from each other. At the time of outputting the result, such numerical value information as the number of reads is output in order to indicate which mutation sequence the result is derived from.

### (2-3) Prediction Generator

Operation of a prediction generator illustrated in FIG. 2 will be described below.

The prediction generator has collected a database where chronological cfDNA analysis data and the state of the subject at the time data acquisition are linked to each other. The prediction generator has also generated a prediction model based on the database. Therefore, it is possible to apply the data of a prediction target person to the prediction model, predict a prognosis, and output the result.

In addition, moreover, the prediction generator may predict recurrence and metastasis in an organ on which a biopsy cannot easily be performed and generate a database for obtaining evidence for performing a biopsy. There are known cases in which malignant tumors metastasize to organs on which a biopsy cannot easily be performed, such as lung, liver, bone, and brain, and become severe.

For example, the following relation is known:
Locations to which breast cancer is likely to metastasize distantly: lung, liver, bone, and brain
Locations to which lung cancer is likely to metastasize distantly: brain, bone, other lung, adrenal gland, liver

Specifically, for example, the prediction generator outputs the possibility of a metastasis according to chronological examinations of blood after the removal of a primary tumor. More specifically, at each examination and upon observation of chronological changes over multiple examinations, a DNA analysis of the subject is performed in reference to a primary tumor tissue of the subject and patterns of other persons in a database where ctDNA analysis results are similar to each other, and the probability of a prognostic metastasis is estimated by a comparison with a subject group in the same category, thereby prompting performing a biopsy on other organs at appropriate times to contribute to early detection of recurrence and metastasis.

### (2-4) Others

Note that the present analysis example 1 is addressed to an analysis process based on genetic mutations. The present embodiment is not limited to such an analysis process. For example, it is possible to generate a target cocktail for a target methylation analysis. Moreover, there may be generated cocktails relating to Tumor mutational burden (TMB), Microsatellite instability high (MSI-H), and gene copy number variation (CNV).

### (3) Flow Examples of Analysis Example 1

Flow examples of the analysis example 1 will be described in detail below.

### (3-1) Flow Example 1

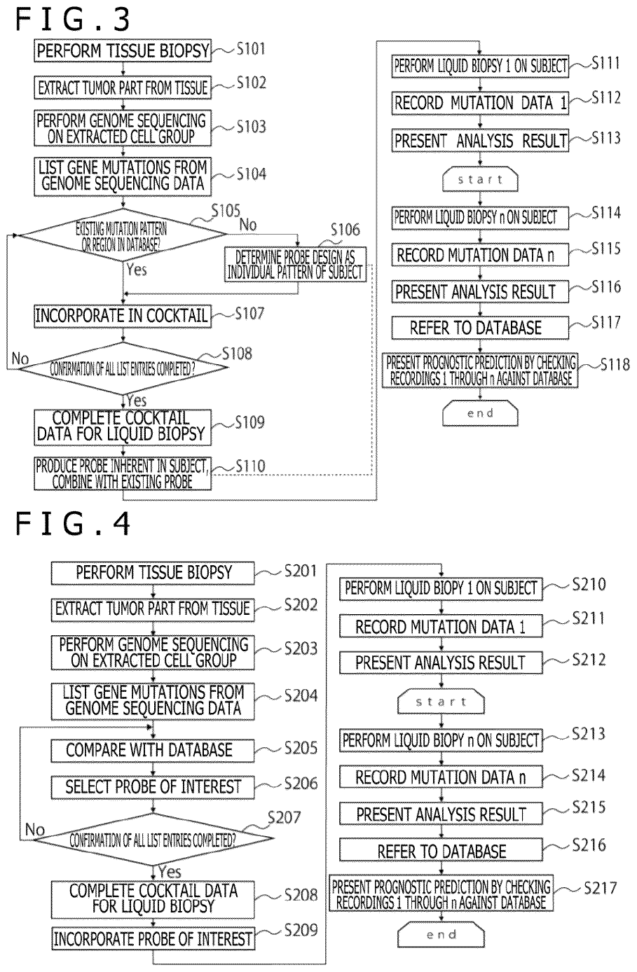

FIG. 3 is a flowchart illustrating a flow example 1 of the analysis example 1. FIG. 3 illustrates a flow example based on the assumption that a database is incomplete and a cocktail is an actual probe. First, an examination is performed on a tumor tissue (S101), and a tumor part is extracted from the obtained tissue (S102). The tumor part may be extracted, for example, by a method of cutting the tumor part from a block, preparing a tissue slide and cutting the tumor part therefrom by way of laser microdissection, or breaking the tumor part into pieces and enriching them by way of flow cytometry. After S102, mutational information regarding genes of the tumor tissue is further acquired in reference to the examination of the tumor tissue. Specifically, for example, genome sequencing is performed on the extracted cell group (S103). The genome sequencing may, for example, be cell bulk sequencing, single-cell genome sequencing, or the like. A spatial transcriptome analysis may be conducted on a tissue section to acquire mutational information.

After S103, information concerning the one or multiple genetic mutations is listed in reference to the mutational information. Specifically, the information concerning genetic mutations is listed from the data regarding the genome sequence obtained in S103 (S104). Then, it is determined whether or not the genetic mutation represents a mutation pattern or region existing in the database (S105). If the genetic mutation represents a mutation pattern or region existing in the database, then it is incorporated as it is into a cocktail (S107). On the other hand, if the genetic mutation does not represent a mutation pattern or region existing in the database, then a design of a probe as a pattern inherent in the subject is determined (S106) and incorporated into a cocktail (S107).

After S107, it is determined whether the confirmation of all the list entries has been completed or not (S108). If completed, then pieces of cocktail data for a blood examination are generated and completed (S109). On the other hand, if the confirmation of all the list entries has not been completed, the processing goes back to step S105, and S105 through S108 (S106 is carried out if necessary) are repeated until the confirmation of all the list entries is completed.

After S109, the probe inherent in the subject that has been determined in S106 is produced, and the produced probe and existing probes are combined (S110). Incidentally, the number of probe cocktails is not limited to any particular value, though it may be specified herein as ranging from 100 to 100,000 or 500 to 5,000, for example. After S110, a first blood examination is conducted on the subject (S111), the pieces of data regarding the genetic mutations are recorded (S112), and the analysis result is presented (S113).

After S113, chronological examinations of blood are conducted to acquire chronological information concerning the one or multiple genetic mutations. Specifically, blood examinations are periodically conducted on the subject (S114), the pieces of data regarding genetic mutations are recorded from the results of the blood examinations (S115), and the analysis result is presented (S116). After S116, an accumulated database is referred to (S117), and the information concerning the first through nth recordings is compared with the database, presenting a prognostic prediction for the subject (S118). Then, S114 through S118 are repeated to acquire chronological information concerning genetic mutations. Note that, when the database is to be referred to, since pieces of data detected by a newly added probe may occasionally be low in reliability, they may be processed by a low level of weighting, for example.

### (3-2) Flow Example 2

FIG. 4 is a flowchart illustrating a flow example 2 of the analysis example 1. FIG. 4 illustrates a flow example based on the assumption that an existing database and retained probes cover almost all genetic mutations (particularly, genetic mutations relating to malignant tumors) and a cocktail is an actual probe. First, an examination of the tumor tissue is performed (S201), and a tumor part is extracted from the obtained tissue (S202). The tumor part may be extracted, for example, by a method of cutting the tumor part from a block, preparing a tissue slide and cutting the tumor part therefrom by way of laser microdissection, or breaking the tumor part into pieces and enriching them by way of flow cytometry. After S202, mutational information regarding genes of the tumor tissue is further acquired in reference to the examination of the tumor tissue. Specifically, for example, genome sequencing is performed on the extracted cell group (S203). The genome sequencing may, for example, be cell bulk sequencing, single-cell genome sequencing, or the like.

After S204, a probe of interest is selected by referring to a generated database, in reference to the mutational information. Specifically, the mutational information obtained from the examination of the tumor tissue and the existing database are compared with each other (S205), and a probe of interest is selected (S206). After S206, it is determined whether the confirmation of all the list entries has been completed or not (S207). If completed, then pieces of cocktail data for a blood examination are completed (S208). On the other hand, if the confirmation of all the list entries has not been completed, the processing goes back to step S205, and S205 through S207 are repeated until the confirmation of all the list entries is completed.

After S208, the probe of interest selected in S206 is incorporated (S209). After S209, a first blood examination is conducted on the subject (S210), the pieces of data regarding the genetic mutations are recorded (S211), and the analysis result is presented (S212). After S212, chronological examinations of blood are conducted to acquire chronological information concerning the one or multiple genetic mutations. Specifically, blood examinations are periodically conducted on the subject (S213), the pieces of data regarding genetic mutations are recorded from the results of the blood examinations (S214), and the analysis result is presented (S215). After S215, an accumulated database is referred to (S216), and the information concerning the first through nth recordings is compared with the database, presenting a prognostic prediction for the subject (S217). Then, S213 through S217 are repeated to acquire chronological information concerning genetic mutations.

### (3-3) Flow Example 3

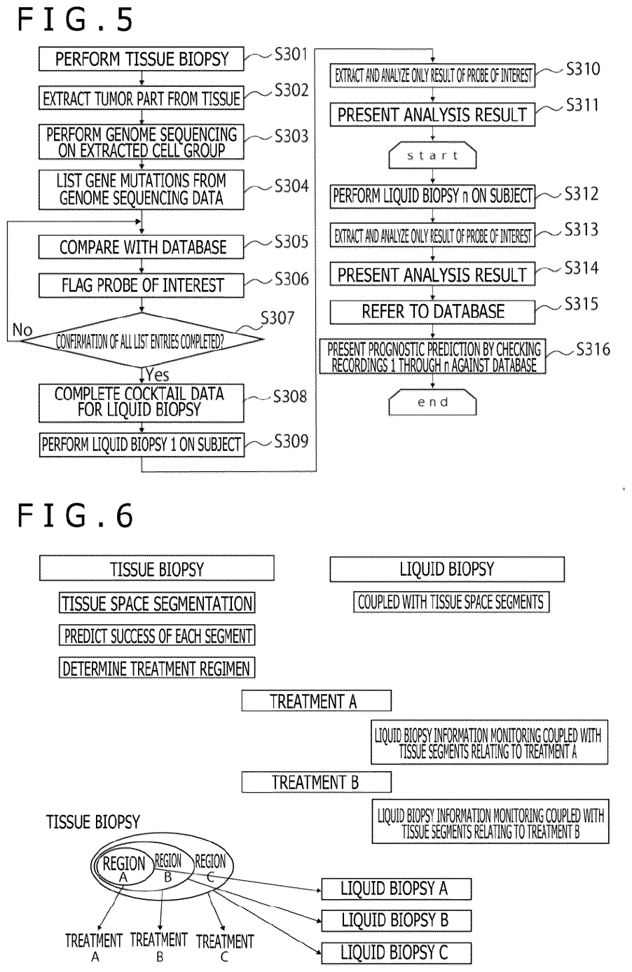

FIG. 5 is a flowchart illustrating a flow example 3 of the analysis example 1. FIG. 5 illustrates a flow example based on the assumption that an existing database and retained probes cover almost all genetic mutations (particularly, genetic mutations relating to malignant tumors) and a cocktail includes only data. First, an examination of the tumor tissue is performed (S301), and a tumor part is extracted from the obtained tissue (S302). The tumor part may be extracted, for example, by a method of cutting the tumor part from a block, preparing a tissue slide and cutting the tumor part therefrom by way of laser microdissection, or breaking the tumor part into pieces and enriching them by way of flow cytometry. After S302, mutational information regarding genes of the tumor tissue is further acquired in reference to the examination of the tumor tissue. Specifically, for example, genome sequencing is performed on the extracted cell group (S303). The genome sequencing may, for example, be cell bulk sequencing, single-cell genome sequencing, or the like.

After S303, information concerning the one or multiple genetic mutations is listed in reference to the mutational information. Specifically, the information concerning genetic mutations is listed from the data regarding the genome sequence obtained in S303 (S304). After S304, the mutational information obtained from the examination of the tumor tissue and the existing database are compared with each other (S205), and a probe of interest is selected and flagged (S306). After S306, it is determined whether the confirmation of all the list entries has been completed or not (S307). If completed, then pieces of cocktail data for a blood examination are completed (S308). On the other hand, if the confirmation of all the list entries has not been completed, the processing goes back to step S305, and S305 through S307 are repeated until the confirmation of all the list entries is completed.

After S308, only the result of the flagged probe of interest is extracted and analyzed. Specifically, a first blood examination is conducted on the subject (S309), only the result of the probe of interest is extracted and analyzed (S310), and the analysis result is presented (S311). After S311, chronological blood examinations are conducted to acquire chronological information concerning the one or multiple genetic mutations. Specifically, blood examinations are periodically conducted on the subject (S312), only the result of the probe of interest is extracted from the result of the blood examinations and analyzed (S313), and the analysis result is presented (S314). After S314, an accumulated database is referred to (S315) and the information concerning the first through nth recordings is compared with the database, presenting a prognostic prediction for the subject (S316). Then, S312 through S316 are repeated to acquire chronological information concerning genetic mutations.

### (4) Analysis Example 2

It is known that a malignant tumor (cancer) is caused by genome mutation, and the cancerization brings about further genome mutation, developing resistance to drugs such as anticancer drugs. Since genome mutation occurs upon cell proliferation that accompanies cell division, the genome mutation becomes spatially diverse in tumor cells proliferated from tumor cells in an early stage. In other words, spatially diverse tumor tissues that have different drug resistance properties are produced, causing spatiotemporal tumor diversity.

Further, as a tumor tissue has spatial diversity, in order to monitor the success of a drug such as an anticancer drug, it is desirable to assess changes in the spatial diversity after the drug has been administered. However, performing a biopsy on the tumor tissue many times on a subject during treatment is highly invasive to the subject. By contrast, measuring biomarkers in a blood examination called a liquid biopsy is less invasive than conducting a biopsy on a tumor tissue, though it lacks spatial information of the tumor tissue.

According to the present analysis example 2, a solution to these problems is proposed.

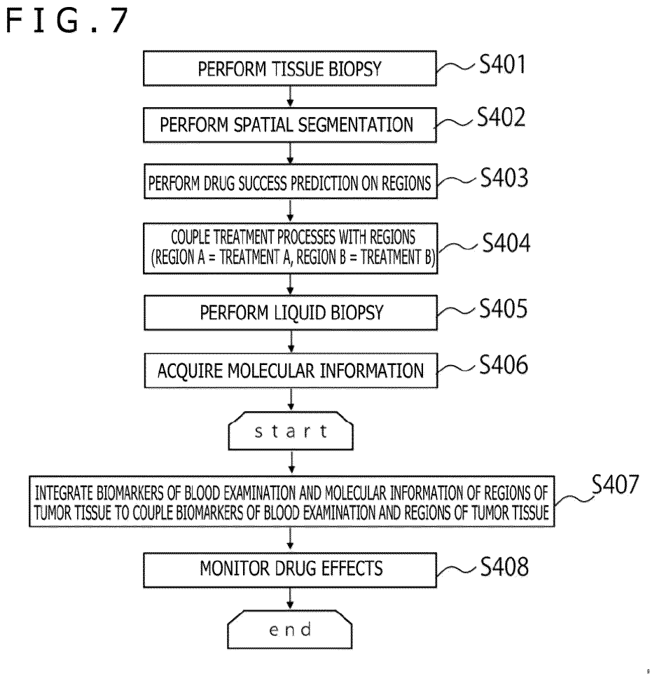

FIG. 6 is a diagram illustrating an example of a concept according to an analysis example 2. Further, FIG. 7 is a flowchart illustrating a flow example of the analysis example 2. First, for an examination of a tumor tissue, one or multiple regions are established and coupled to biomarkers in blood examinations. Specifically, the examination of the tumor tissue is carried out (S401), and special segmentation is performed on the biopsy sample to establish one or multiple regions (S402; in FIG. 6, a region A, a region B, and a region C are established). Then, prediction of the success of a drug such as an anticancer drug is performed on the regions in reference to molecular information (for example, genome, mRNA, protein, and the like) (S403). Treating methods (in FIG. 6, treatment A, treatment B, and treatment C) are coupled to the respective regions (S404; in FIG. 6, the region A=the treatment A, the region B=the treatment B).

Then, using the coupled biomarkers, the states of the tumor in the regions are predicted. Specifically, another blood examination is carried out (S405), and molecular information is acquired (S406). Then, biomarkers of the blood examination and molecular information of regions of the tumor tissue are integrated to couple the biomarkers of the blood examination and the regions of the tumor tissue (S407). In a case of determining a treatment policy from a spatial distribution of the regions of the tumor tissue, it is thus possible to monitor the effect of the drug by using the regions of the tumor tissue and the biomarkers of the blood examination that are coupled to the regions (S408). For example, as illustrated in FIG. 6, in the treatment A, the effect of the drug is monitored using the information concerning the blood examination that is coupled to the region (region A) of the tumor tissue relative to the treatment A, and in the treatment B, the effect of the drug is monitored using the information concerning the blood examination that is coupled to the region (region B) of the tumor tissue relative to the treatment B. Further, the monitoring process is repeated as many times as the number of combinations of the regions of the tumor tissue and the biomarkers of the blood examination that are established in the treatment process (that is, S407 and S408 are repeated).

Note that an examination of the tumor tissue may not necessarily be able to acquire the tumor tissue in its entirety, resulting in a need to estimate the overall tumor from an acquired region. In such a case, according to the analysis example 2, it is possible to estimate the sizes of the corresponding regions of the tumor tissue from the amounts of the biomarkers in the blood examination and to estimate spatial information (for example, to estimate that the tumor tissue exists in a place that is so spatially accessible that the treatment is successful) from the success of the corresponding treatment.

### (5) Analysis Example 3

In a case where prediction is to be made with regard to a tumor with use of multimodal data, many measurement items are involved, and determination standards are correspondingly multifaceted. The many measurement items lead to an increase in man-hours and a higher examination cost. Further, the multifaceted determination standards mean that whereas the assessments of some items may exceed their determination standards, the assessments of other items may fall short of their determination standards, with the result that an overall assessment index may be lower than its determination standard.

Specifically, for example, if pieces of multimodal data are used in a companion diagnostics that is carried out in selecting a drug to be administered, then a cause that makes or does not make a drug a drug administration target can depend on various factors. Hence, if pieces of multimodal data are required by each examination, the number of examinations increases, giving rise to the above problem of increase in man-hours and a higher examination cost. Moreover, an increase in the number of times that a tumor tissue is acquired places an increased burden on the subject.

Therefore, according to the present analysis example 3, a process of solving these problems is proposed.

The present analysis example 3 represents a process of efficiently selecting examination items at the time when an examination is performed to give a subject who has once not become a drug administration target an opportunity to be given a drug. For example, there is an application for determining that the subject is a target to which a desired drug is to be administered if the assessments of a predetermined number of items among multiple biomarkers exceed their determination standards. In this case, if the assessments of the predetermined number of items do not exceed their determination standards, then the subject does not become a target to which a desired drug is to be administered.

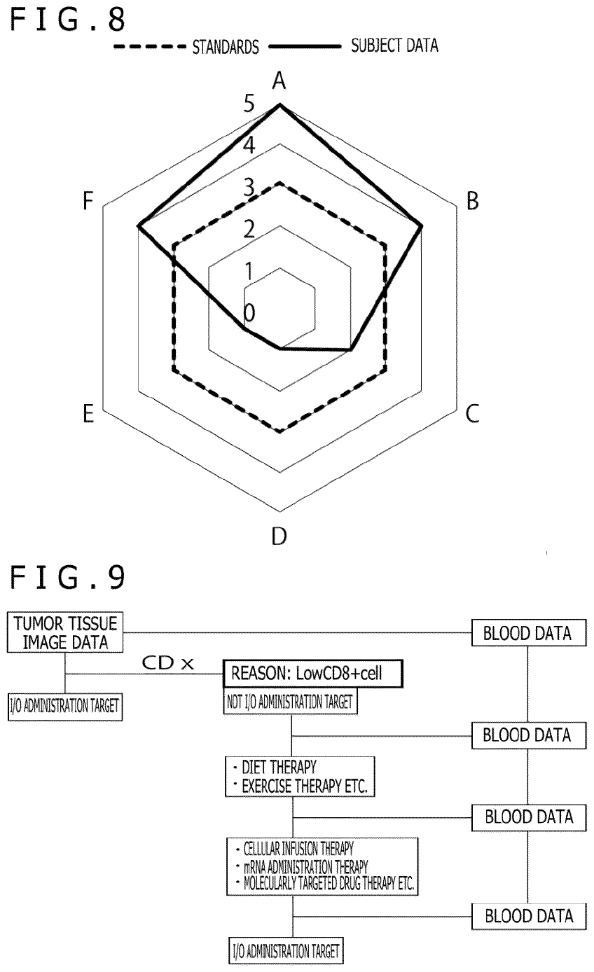

FIG. 8 is a diagram illustrating an example of biomarkers obtained from a subject by an examination of a tumor tissue and an example of determination standards for an administration target. In FIG. 8, there are six biomarkers A through F obtained from a tissue biopsy, and each standard value is set to 3. If four or more biomarkers among the six biomarkers A through F exceed the standard value, then the drug becomes an administration target.

In FIG. 8, however, since only the three biomarkers A, B, and F exceed the standard value, the drug is determined as not being an administration target, and the drug does not become an administration target. In this case, if any one of the remaining biomarkers C, D, and E exceeds the standard value, then the drug becomes an administration target. Consequently, the biomarkers C, D, and E are preferentially monitored to pay attention to whether or not they exceed the standard value, so that it is efficiently determined whether a desired drug that has once been determined as not being an administration target can become an administration target.

Specifically, according to the present analysis example 3, blood examinations are chronologically performed to find out about a particular biomarker among one or multiple biomarkers that is selected based on an examination of a tumor tissue (tissue biopsy), to allow a desired drug that has once not become an administration target to be reviewed again. According to the present analysis example 3, the particular biomarker should preferably be a biomarker that has fallen out of the standards for an administration target in a first or second or subsequent blood examination, as described above. In the above example, the biomarkers C, D, and E represent the particular biomarkers.

Note that numerical values obtained from a tissue biopsy and numerical values obtained from a blood biopsy may possibly be different from each other in terms of their absolute values. In that case, the numerical values may be determined from relative values of data of the blood biopsy. Further, the chronological examinations of blood referred to above may be used as determination standards for determining whether a tissue biopsy is to be performed again or not. Moreover, it is possible to determine whether a drug can be included as an administration target or not from only data obtained by monitoring some measurement items of a blood biopsy.

Further, with regard to multimodal data, there is an occasion in which each measurement item has no determination standard value but an overall assessment index such as clustering. In such an occasion, an item to be monitored may be determined depending on the degree to which a biomarker contributes to an assessment index. An item with a large degree of contribution may be an item where an assessment index changes greatly, for example. Furthermore, an item that is easily expected to improve may preferentially be selected from the state of the subject.

FIG. 9 is a diagram illustrating an example of a concept of an application of the analysis example 3. FIG. 9 illustrates an example in which treatment is recommended to increase a measurement value for an item whose determination standard has been lowered. For example, if a tumor tissue is determined as not being a target to which a desired drug is to be administered because of falling short of immune cells such as CD8+, for example, then treatment for increasing immune cells that have been determined as falling short of is recommended. Specifically, a diet therapy, an exercise therapy, a cellular infusion therapy, an mRNA administration therapy, or a molecularly targeted drug therapy, for example, may be recommended. While the subject is being treated with a recommended therapy, it is also possible to continue to acquire chronological data of blood examinations and to determine from time to time whether the tumor tissue can be a target to which the desired drug is to be administered.

### (6) Analysis Example 4

As described above, since an examination of a tumor tissue is highly invasive, it is burdensome on the subject, and the number of times that it is conducted cannot be increased in practice.

For this reason, the present analysis example 4 proposes a process of predicting missing data by integrating an examination of a tumor tissue and chronological examinations of blood.

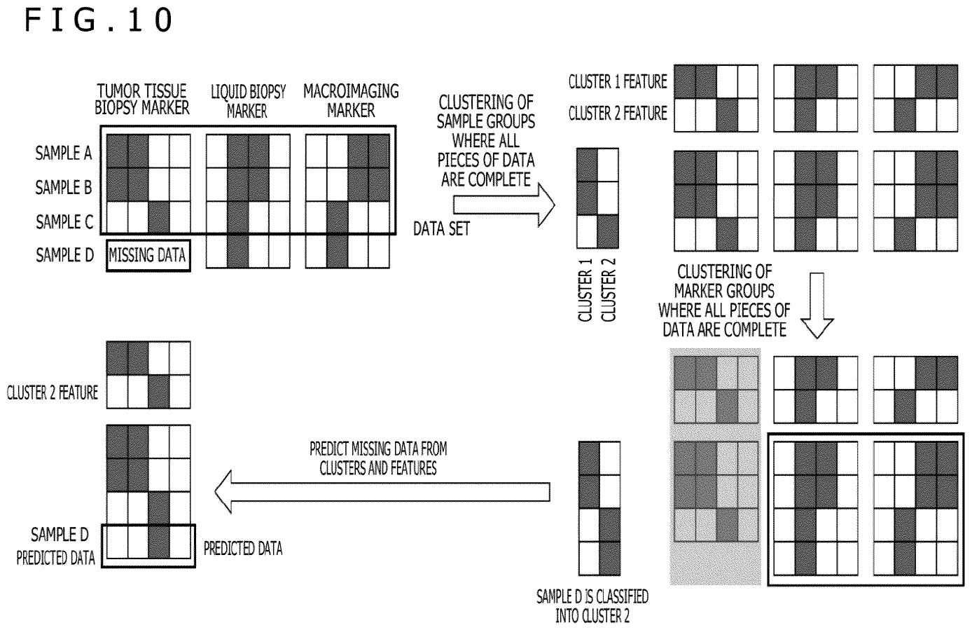

FIG. 10 is a diagram illustrating an example of a concept according to the analysis example 4. In FIG. 10, there are produced matrixes including rows that represent subjects (in FIG. 10, a sample A, a sample B, a sample C, and a sample D) and columns that represent measurement values of respective biomarker measurement modes (in FIG. 10, a tumor tissue biopsy marker, a liquid biopsy marker, and a macroimaging marker) for the respective biomarker measurement modes. The rows that represent the subjects are arranged in a common order between the modes.

Then, a data set of clusters and features are calculated in reference to an information group regarding one or multiple biomarkers linked to tumor tissues and blood. Specifically, as illustrated in FIG. 10, for example, using pieces of data regarding a subject group in which the measurement values of all biomarker measurement modes of the tumor tissues and the blood are complete (In FIG. 10 the sample A, the sample B, the sample C), a dimensionality compression having the information concerning the multiple biomarkers is performed (the multiple matrixes are simultaneously decomposed) by Joint Non-negative Matrix Factorization (jNMF), for example, and the result of the dimensionality compression is clustered, thereby calculating and holding a data set of clusters and features.

Then, based on the data set, information regarding one or multiple biomarkers that are missing is estimated. Specifically, a matrix in which a subject sample for which not all measurement values of all biomarker measurement modes are complete (for example, measurement values of a highly invasive tumor tissue biopsy marker, i.e., a tumor tissue biopsy marker of a sample D in FIG. 10) is added to the row of subjects is produced with only biomarker measurement modes whose measurement values are complete, and clustering is performed by jNMF, for example, thereby obtaining clusters and features. Further, by referring to the data set from a cluster (in FIG. 10, a cluster 2) including the added subject (in FIG. 10, the sample D), measurement values of a missing biomarker measurement mode (in FIG. 10, predicted data of the sample D) can be estimated from the features of the clusters of the data set.

### (7) Analysis Example 5

It is difficult for the present technology to analyze the clonality of a T cell from a tumor tissue sample while keeping its spatial information. Meanwhile, it is possible to detect the clonality of a T cell by performing a single cell repertoire analysis (single cell TCR analysis) on a blood sample. Furthermore, a tissue of origin can be estimated with high accuracy by a methylation analysis of CEDNA and ctDNA in a blood sample.

Hence, according to the present analysis example 5, there are proposed an effective treatment method and a process of estimating an accurate state of a tumor tissue by integrating the information of both samples.

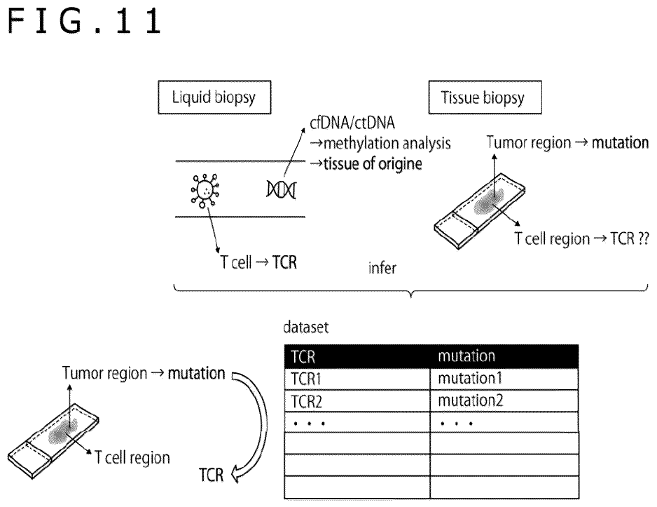

FIG. 11 is a diagram illustrating an example of a concept according to the analysis example 5. According to the analysis example 5, a database of mutation-TCR is constructed based on an examination of a tumor tissue and chronological examinations of blood. Specifically, somatic mutations of a tumor tissue sample and information of TCR in a blood sample are integrated to construct a database of mutation-TCR, making it possible to estimate from one information other information with high accuracy. Consequently, both pieces of information can be used to generate CAR-T or TCR-T, leading to a cell immunotherapy.

Incidentally, “TCR” referred to above represents glycoprotein that occurs in T cells and is known as a receptor that operates when a cell recognizes a cancer antigen and the like. In addition, “CAR” refers to a chimera antigen and is known as a receptor that is artificially produced in such a manner as to be bonded to a particular membrane protein. Moreover, “CAR-T” or “TCR-T” refers to treatment in which CAR genes or TCR genes that specifically recognize cancer cells are introduced ex vivo into T cells sampled from a subject (particularly, a person affected with a malignant tumor), proliferated by way of cultivation, and then infused into the subject.

Further, using the information in the database described above, it is expected that the present analysis example 5 is useful in an antibody therapy using a bi-specific antibody or the like, making it possible to realize an individualized immunotherapy.

Moreover, in cfDNA and ctDNA, genome-like closed chromatin regions are easy to analyze. However, in blood samples, there is no guarantee that all closed chromatin regions will be analyzed. Meanwhile, from tumor tissue samples, open chromatin regions that are decomposed and difficult to analyze in blood samples can be analyzed by use of ATAC (Assay for Transposase-Accessible Chromatin)-seq. Furthermore, histone modification, transcription factor, and the like in closed chromatin regions can be analyzed using ChiP-seq, CUT & tag (Cleavage Under Targets and Tagmentation), and CU & RUN (Cleavage Under Target & Release Using Nuclease), for example.

According to the present analysis example 5, therefore, the information concerning at least closed chromatin regions can be acquired based on an examination of a tumor tissue and chronological examinations of blood. Moreover, closed chromatin regions can be specified with higher accuracy by use of the above process. Furthermore, the information concerning open chromatin regions specified from a tumor tissue sample and the information concerning histone modification, transcription factor, and the like in closed chromatin regions can be supplemented and added to estimate the stage of the patient that is predicted from closed chromatin regions in a blood sample.

### 2. Second Embodiment (a System 1 for Analyzing a Tumor)

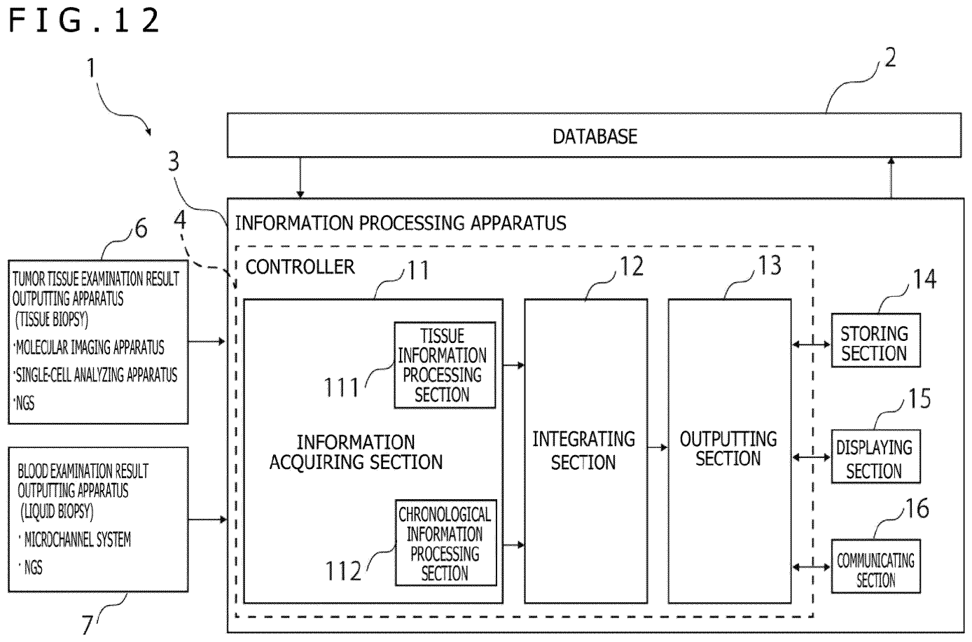

FIG. 12 is a block diagram illustrating an example of a system 1 for analyzing a tumor. The system 1 for analyzing a tumor according to the present embodiment may have at least an information processing apparatus 3 that includes a controller 4 including an information acquiring section 11, an integrating section 12, and an outputting section 13, a storing section 14, a displaying section 15, and a communicating section 16, a tumor tissue examination result outputting apparatus 6, and a blood examination result outputting apparatus 7. The system 1 for analyzing a tumor according to the present embodiment may include a database 2 or the like when necessary.

The components of the system 1 for analyzing a tumor according to the present embodiment will be described in detail hereinbelow.

### (1) Information Acquiring Section 11

The information acquiring section 11 acquires information regarding an examination of a tumor tissue and chronological examinations of blood. Specifically, the information acquiring section 11 acquires examination data of a tumor tissue acquired using a molecular imaging apparatus, a single-cell analyzing apparatus, or an NGS, for example, and chronological examination data of blood acquired using a microchannel system or an NGS, for example, from the tumor tissue examination result outputting apparatus 6 or the blood examination result outputting apparatus 7 that is connected to the information acquiring section 11 via a network. Alternatively, the information acquiring section 11 may acquire the information that is directly input from the user via a user interface (not depicted).

According to the present embodiment, the information acquiring section 11 includes a tissue information processing section 111 and a chronological information processing section 112. The tissue information processing section 111 performs every analysis in reference to the examination data of the tumor tissue and converts analysis results into data. Note that it is possible for the tissue information processing section 111 to perform feedback control on the tumor tissue examination result outputting apparatus 6 in reference to the data. Similarly, the chronological information processing section 112 performs every analysis in reference to the chronological examination data of the blood and converts analysis results into data. It is possible for the chronological information processing section 112 to perform feedback control on the blood examination result outputting apparatus 7 in reference to the data.

The information acquiring section 11 may store in advance programs for performing various analyses or may, as needed, download necessary programs from the storing section 14, to be described later, or the database 2 in a cloud system and execute the downloaded programs.

The information acquiring section 11 may be implemented as an information acquiring apparatus such as a general-purpose computer and may include a CPU, a RAM, and a ROM. Further, the information acquiring section 11 may be housed in the casing of the information processing apparatus 3 as illustrated in FIG. 12 or may be provided outside of the casing of the information processing apparatus 3. Further, various processing operations or various functions of the information acquiring section 11 may be implemented by a server computer or a cloud system that is connected via a network.

### (2) Integrating Section 12

The integrating section 12 integrates information from the information acquiring section 11 and generates data regarding a tumor. Specifically, the integrating section 12 integrates the analysis result data based on the examination of the tumor tissue and the analysis result data based on the chronological examinations of the blood that have been acquired from the information acquiring section 11, and performs the processing operations described in the analysis examples 1 through 5 of “1. First embodiment (a method of analyzing a tumor)” described above, for example.

The integrating section 12 may store in advance programs for performing various analyses or may, as needed, download necessary programs from the storing section 14, to be described later, or the database 2 in the cloud system and execute the downloaded programs.

As with the information acquiring section 11, the integrating section 12 may be implemented as an information acquiring apparatus such as a general-purpose computer and may include a CPU, a RAM, and a ROM. Further, the integrating section 12 may be housed in the casing of the information processing apparatus 3 as illustrated in FIG. 12 or may be provided outside of the casing of the information processing apparatus 3. Further, various processing operations or various functions of the integrating section 12 may be implemented by a server computer or a cloud system that is connected via a network.

### (3) Outputting Section 13

The outputting section 13 acquires the data regarding the tumor that has been generated by the integrating section 12 and outputs the acquired data as digital data out of the controller 4. Specifically, the acquired data is output to the storing section 14, the displaying section 15, and the communicating section 16, for example. According to the present embodiment, however, the destination to which the acquired data is output is not limited to these sections. Specifically, the outputting section 13 may output the acquired data to a server computer or a cloud system that is connected via a network.

### (5) Storing Section 14

The storing section 14 includes, for example, a nonvolatile storage medium such as a hard disk drive or a flash memory and a storage controller for controlling the storage medium to write and read data. The storing section 14 stores not only all of the pieces of data regarding the tumor that have been generated by the integrating section 12, but also the original data (the examination data of the tumor tissue and the chronological examination data of the blood) used to generate the data regarding the tumor. Further, the storing section 14 may store the programs used to implement the information acquiring section 11 and the integrating section 12. Moreover, the storing section 14 may store in advance information regarding the subject individual and data obtained thus far regarding the subject.

### (5) Displaying Section 15

The displaying section 15 presents the data output from the outputting section 13 to the user. Note that the displaying section 15 is not limited to any particular devices or means. According to the present embodiment, moreover, the data output from the outputting section 13 may be presented to the user by being projected as an image by a projector or printed by a printer.

### (6) Communicating Section 16

The communicating section 16 presents the data output from the outputting section 13 onto a network. Means for presenting the data onto the network may be implemented as a communication card for use with a wired or wireless LAN (Local Area Network), LTE (Long Term Evolution), Bluetooth (registered trademark), or WUSB (Wireless USB), for example.

### (7) Tumor Tissue Examination Result Outputting Apparatus 6

The tumor tissue examination result outputting apparatus 6 outputs at least examination data of a tumor tissue to the tissue information processing section 111 in the information acquiring section 11. The tumor tissue examination result outputting apparatus 6 may output the examination data via a user interface provided in or connected to the tumor tissue examination result outputting apparatus 6 or may output therefrom directly to the tissue information processing section 111 via a network without the intervention of the user.

Further, the tumor tissue examination result outputting apparatus 6 may not only output the examination data, but also have a function to perform examinations themselves. Moreover, though the tissue information processing section 111 described above performs every analysis in reference to examination data of a tumor tissue and converts analysis results into data, the present embodiment is not limited to such a feature, and the tumor tissue examination result outputting apparatus 6 may perform every analysis in reference to examination data of a tumor tissue. The tumor tissue examination result outputting apparatus 6 may include an apparatus capable of tissue biopsies, and may specifically include a molecular imaging apparatus, a single-cell analyzing apparatus, or an NGS, for example.

### (8) Blood Examination Result Outputting Apparatus 7

The blood examination result outputting apparatus 7 outputs at least examination data of blood (including chronological examination data of blood) to the chronological information processing section 112 in the information acquiring section 11. The blood examination result outputting apparatus 7 may output the examination data via a user interface provided in or connected to the blood examination result outputting apparatus 7 or may output therefrom directly to the chronological information processing section 112 via a network without the intervention of the user.

Further, the blood examination result outputting apparatus 7 may not only output the examination data, but also have a function to perform examinations themselves. Moreover, though the chronological information processing section 112 described above performs every analysis in reference to examination data of blood and converts analysis results into data, the present embodiment is not limited to such a feature, and the blood examination result outputting apparatus 7 may perform every analysis in reference to examination data of blood. The blood examination result outputting apparatus 7 may include an apparatus capable of liquid biopsies, and may specifically include a microchannel system or an NGS, for example.

### (9) Database 2

The database 2 is connected to the information processing apparatus 3 via a network, organizes every pieces of data acquired or analyzed by the information processing apparatus 3 (including data analyzed by the integrating section 12), and stores the organized data into a device including a known storage medium, for example.

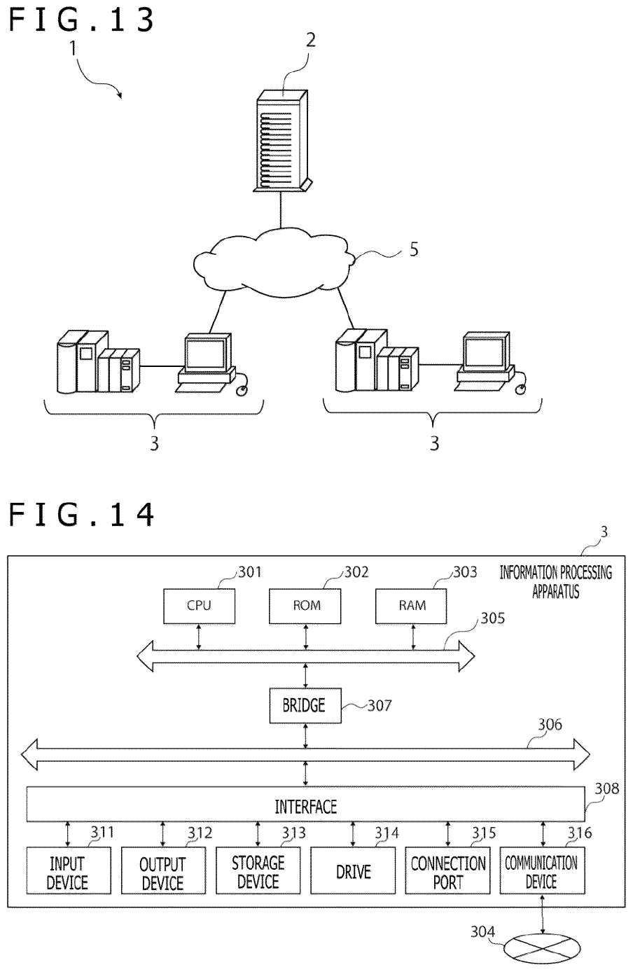

FIG. 13 is a schematic conceptual diagram illustrating a modification of the database 2. According to the present embodiment, as illustrated in FIG. 13, the database 2 may be provided on a cloud system 5 for storing a vast amount of data. The stored data may be shared by information processing apparatuses 3 for providing analyses of higher quality. Note that, in FIG. 13, the tumor tissue examination result outputting apparatus 6 and the blood examination result outputting apparatus 7 are omitted from illustration.

### (10) Example of a Hardware Configuration of an Information Processing Apparatus 3

FIG. 14 is a block diagram illustrating an example of a hardware configuration of the information processing apparatus 3. Various processing operations of the information processing apparatus 3 are, for example, implemented by cooperation of software and hardware to be described below.

As illustrated in FIG. 14, the information processing apparatus 3 includes a CPU (Central Processing Unit) 301, a ROM (Read Only Memory) 302, a RAM (Random Access Memory) 303, and a host bus 305. Moreover, the information processing apparatus 3 includes a bridge 307, an external bus 306, an interface 308, an input device 311, an output device 312, a storage device 313, a drive 314, a connection port 315, and a communication device 316. Incidentally, the information processing apparatus 3 may have a processing circuit such as a DSP or an ASIC, for example, instead of or together with the CPU 301.

The CPU 301 functions as an arithmetic processing device and a control device and controls all operations in the information processing apparatus 3 according to various programs. Further, the CPU 301 may be a microprocessor. The ROM 302 stores programs, calculation parameters, and the like that are used by the CPU 301. The RAM 303 temporarily stores programs used by the CPU 301 in its processing sequences and parameters that vary as appropriate in its processing sequences, for example. The CPU 301 can realize at least the controller 4 of the information processing apparatus 3, for example.

The CPU 301, the ROM 302, and the RAM 303 are interconnected by the host bus 305 that includes a CPU bus and the like. The host bus 305 is connected to the external bus 306 such as a PCI (Peripheral Component Interconnect/Interface) bus, for example via the bridge 307. Note that, according to the present embodiment, the host bus 305, the bridge 307, and the external bus 306 may not necessarily be separate from each other, and may have their functions incorporated in a single bus.

The input device 311 may be implemented by a device to which the user can input information, such as a mouse, a keyboard, a touch panel, buttons, a microphone, switches, or levers, for example. Alternatively, the input device 311 may include a remote control device using infrared rays or other radio waves, or may be an externally connected device such as a cellular phone or a PDA, for example, compatible with the information processing apparatus 3 for its operation. Further, the input device 311 may include an input control circuit that generates input signals in reference to information input by the user via the inputting means described above and outputs the generated input signals to the CPU 301, for example. The user operates the input device 311 to input various kinds of data to the information processing apparatus 3 and instruct the information processing apparatus 3 to perform processing operations. The input device 311 can realize at least a user interface (not depicted) of the information processing apparatus 3, for example.

The output device 312 is formed as a device capable of indicating acquired information visually or auditorily to the user. Such a device may include a display device such as a CRT display device, a liquid crystal display device, a plasma display device, an EL display device, or lamps, an acoustic output device such as a speaker or headphones, or a printer device, for example. The output device 312 can realize at least the displaying section 15 of the information processing apparatus 3, for example.

The storage device 313 refers to a device for storing data. The storage device 313 is realized by a magnetic storage device such as an HDD, a semiconductor storage device, an optical storage device, or a magnetooptical storage device, for example. The storage device 313 may include a storage medium, a recording device for recording data on the storage medium, a reading device for reading data from the storage medium, and a deleting device for deleting data recorded on the storage medium, for example. The storage device 313 stores programs executed and various kinds of data used by the CPU 301 and various kinds of data acquired from an external source. The storage device 313 can realize at least the storing section 14 of the information processing apparatus 3, for example.

The drive 314 refers to a reader/writer for the storage medium and is incorporated in or externally connected to the information processing apparatus 3. The drive 314 reads information recorded on a removable storage medium mounted therein such as a magnetic disk, an optical disk, a magnetooptical disk, or a semiconductor memory, for example, and outputs the read information to the RAM 303. Moreover, the driver 314 can write information on the removable storage medium.

The connection port 315 refers to an interface to be connected to an external device and functions as a connection port for connection to an external device that is capable of transmitting data via a USB (Universal Serial Bus), for example.

The communication device 316 refers to a communication interface provided by a communication device for connection to the network 304, for example. The communication device 316 may be implemented as a communication card for use with a wired or wireless LAN (Local Area Network), LTE (Long Term Evolution), Bluetooth (registered trademark), or WUSB (Wireless USB), for example. Further, the communication device 316 may refer to a router for optical communication, a router for ADSL (Asymmetric Digital Subscriber Line), or a modem for various types of communication. The communication device 316 can send and receive signals according to a predetermined protocol such as TCP/IP, for example, to and from the Internet or other communication devices.

The network 304 refers to a wired or wireless transmission line for information that is transmitted from a device connected to the network 304. The network 304 may include, for example, the Internet, a public circuit network such as a telephone communication network or a satellite communication network, any of various LANs (Local Area Networks) including Ethernet (registered trademark), and a WAN (Wide Area Network), for example. The network 304 may also include a dedicated circuit network such as IP-VPN (Internet Protocol-Virtual Private Network).

The example of the hardware configuration that can perform the functions of the information processing apparatus 3 has been described above. The components described above may be realized by general-purpose components or may be realized by pieces of hardware that are dedicated to the functions of the components. Consequently, it is possible to suitably change the hardware structures used according to the technical level at the time the present embodiment is reduced to practice.

In addition, it is also possible to produce computer programs for performing the functions of the information processing apparatus 3 described above and install the computer programs in a PC, for example. Moreover, a computer-readable recording medium that is storing such computer programs can be provided. The recording medium may include a magnetic disk, an optical disk, a magnetooptical disk, or a flash memory, for example. Further, the computer programs may be provided via a network rather than by the recording medium, for example.

### 3. Third Embodiment (a Method of Generating Analyzed Data of a Tumor)

A method of generating analyzed data of a tumor according to the present embodiment uses an examination of a tumor tissue and chronological examinations of blood and has the examinations intervene in each other to integrally analyze data from the examinations, thereby generating data regarding the tumor.

In the method of generating analyzed data of a tumor according to the present embodiment, the generated analyzed data refers to data generated in the analysis examples 1 through 5 of the “1. First embodiment (a method of analyzing a tumor)” described above, for example. The analyzed data may be generated by the integrating section 12 of the “2. Second embodiment (a system 1 for analyzing a tumor)” described above, for example. According to the present embodiment, the analyzed data is not limited to data thus generated. Specific analyzed data has been described above and will be omitted from description here.

The method of generating analyzed data of a tumor according to the present embodiment may further acquire mutational information regarding genes of a tumor tissue, based on an examination of the tumor tissue, and list information concerning one or multiple genetic mutations in reference to the mutational information. A specific method of generating data has been described in the analysis example 1 of the “1. First embodiment (a method of analyzing a tumor” and will be omitted from description here.

Further, the method of generating analyzed data of a tumor according to the present embodiment may establish one or multiple regions for an examination of a tumor tissue, couple biomarkers in an examination of blood to the regions, and predict the states of the tumor in the regions by using the coupled biomarkers. A specific method of generating data has been described in the analysis example 2 of the “1. First embodiment (a method of analyzing a tumor” and will be omitted from description here.

In addition, the method of generating analyzed data of a tumor according to the present embodiment may calculate a data set of clusters and features, in reference to an information group regarding one or multiple biomarkers linked to tumor tissues and blood, and estimate information regarding missing one or multiple biomarkers, in reference to the data set. A specific method of generating data has been described in the analysis example 4 of the “1. First embodiment (a method of analyzing a tumor” and will be omitted from description here.

Incidentally, the generated analyzed data may be stored in a removable storage medium such as a magnetic disk, an optical disk, a magnetooptical disk, or a semiconductor memory, for example, and may be uploaded into a database on a cloud system.

Note that the present technology can also take the following configurations.

[1]

A method of analyzing a tumor, including:
acquiring information regarding a tumor by using an examination of a tumor tissue and chronological examinations of blood.
   [2]

The method of analyzing a tumor according to [1], further including:
acquiring mutational information regarding genes of the tumor tissue, in reference to the examination of the tumor tissue.
   [3]

The method of analyzing a tumor according to [2], further including:
listing information concerning one or multiple genetic mutations, in reference to the mutational information.
   [4]

The method of analyzing a tumor according to [3], further including:
acquiring chronological information regarding the one or multiple genetic mutations by the chronological examinations of blood.
   [5]

The method of analyzing a tumor according to [2], further including:
selecting a probe of interest by referring to a database generated in reference to the mutational information.
   [6]

The method of analyzing a tumor according to [5], further including:
flagging the probe of interest.
   [7]

The method of analyzing a tumor according to [6], further including:
extracting and analyzing only a result of the flagged probe of interest.
   [8]

The method of analyzing a tumor according to any one of [1] through [7], further including:
establishing one or multiple regions for the examination of the tumor tissue; and
coupling biomarkers in the an examination of blood to the regions.
   [9]

The method of analyzing a tumor according to [8], further including:
predicting states of the tumor in the regions by using the coupled biomarkers.
   [10]

The method of analyzing a tumor according to any one of [1] through [9], further including:
performing the chronological examinations of the blood with regard to a particular biomarker of the one or multiple biomarkers that is selected according to the examination of the tumor tissue.
   [11]

The method of analyzing a tumor according to [10], in which the particular biomarker is a biomarker that has fallen out of determination standards for an administration target in a first or second or subsequent examination.

[12]

The method of analyzing a tumor according to any one of [1] through [11], further including:
calculating a data set of clusters and features in reference to an information group regarding one or multiple biomarkers linked to tumor tissues and blood.
   [13]

The method of analyzing a tumor according to [12], further including:
estimating information regarding missing one or multiple biomarkers, in reference to the data set.
   [14]

The method of analyzing a tumor according to any one of [1] through [13], further including:
constructing a database of mutation-TCR, in reference to the examination of the tumor tissue and the chronological examinations of the blood.
   [15]

The method of analyzing a tumor according to any one of [1] through [14], further including:
acquiring information concerning at least closed chromatin regions, in reference to the examination of the tumor tissue and the chronological examinations of the blood.
   [16]

A system for analyzing a tumor, including:
an information processing apparatus that includes
   a controller including an information acquiring section for acquiring information regarding an examination of a tumor tissue and chronological examinations of blood, an integrating section for integrating the information from the information acquiring section to generate data regarding the tumor, and an outputting section for outputting the data generated by the integrating section,
   a storing section for storing the data output from the outputting section,
   a displaying section for displaying the data output from the outputting section, and
   a communicating section for presenting the data output from the outputting section onto a network;
a tumor tissue examination result outputting apparatus for outputting a result of the examination of the tumor tissue; and
a blood examination result outputting apparatus for outputting a result of the examinations of the blood.
   [17]

A method of generating analyzed data of a tumor, including:
using an examination of a tumor tissue and chronological examinations of blood and having the examinations intervene in each other to integrally analyze data from the examinations, thereby generating data regarding the tumor.
   [18]

The method of generating analyzed data of a tumor according to [17], further including:
acquiring mutational information regarding genes of the tumor tissue in reference to the examination of the tumor tissue; and
listing information concerning one or multiple genetic mutations in reference to the mutational information.
   [19]

The method of generating analyzed data of a tumor according to [17] or [18], further including:
establishing one or multiple regions for the examination of the tumor tissue;
coupling biomarkers in an examination of blood to the regions; and
predicting states of the tumor in the regions by using the coupled biomarkers.
   [20]

The method of generating analyzed data of a tumor according to any one of [17] through [19], further including:
calculating a data set of clusters and features in reference to an information group regarding one or multiple biomarkers linked to tumor tissues and blood; and
estimating information regarding missing one or multiple biomarkers in reference to the data set.

### REFERENCE SIGNS LIST

1: System for analyzing a tumor
11: Information acquiring section
111: Tissue information processing section
112: Chronological information processing section
12: Integrating section
13: Outputting section
14: Recording section
15: Displaying section
16: Communicating section
2: Database
3: Information processing apparatus
301: CPU
302: ROM
303: RAM
304: Network
305: Host bus
306: External bus
307: Bridge
308: Interface
311: Input device
312: Output device
313: Storage device
314: Drive
315: Connection port
316: Communication device
5: Cloud system
6: Tumor tissue examination result outputting apparatus
7: Blood examination result outputting apparatus

1. A method of analyzing a tumor, comprising:
acquiring information regarding a tumor by using an examination of a tumor tissue and chronological examinations of blood.

2. The method of analyzing a tumor according to claim 1, further comprising:
acquiring mutational information regarding genes of the tumor tissue, in reference to the examination of the tumor tissue.

3. The method of analyzing a tumor according to claim 2, further comprising:
listing information concerning one or multiple genetic mutations, in reference to the mutational information.

4. The method of analyzing a tumor according to claim 3, further comprising:
acquiring chronological information regarding the one or multiple genetic mutations by the chronological examinations of blood.

5. The method of analyzing a tumor according to claim 2, further comprising:
selecting a probe of interest by referring to a database generated in reference to the mutational information.

6. The method of analyzing a tumor according to claim 5, further comprising:
flagging the probe of interest.

7. The method of analyzing a tumor according to claim 6, further comprising:
extracting and analyzing only a result of the flagged probe of interest.

8. The method of analyzing a tumor according to claim 1, further comprising:
establishing one or multiple regions for the examination of the tumor tissue; and
coupling biomarkers in the an examination of blood to the regions.

9. The method of analyzing a tumor according to claim 8, further comprising:
predicting states of the tumor in the regions by using the coupled biomarkers.

10. The method of analyzing a tumor according to claim 1, further comprising:
performing the chronological examinations of the blood with regard to a particular biomarker of the one or multiple biomarkers that is selected according to the examination of the tumor tissue.

11. The method of analyzing a tumor according to claim 10, wherein the particular biomarker is a biomarker that has fallen out of determination standards for an administration target in a first or second or subsequent examination.

12. The method of analyzing a tumor according to claim 1, further comprising:
calculating a data set of clusters and features in reference to an information group regarding one or multiple biomarkers linked to tumor tissues and blood.

13. The method of analyzing a tumor according to claim 12, further comprising:
estimating information regarding missing one or multiple biomarkers, in reference to the data set.

14. The method of analyzing a tumor according to claim 1, further comprising:
constructing a database of mutation-TCR, in reference to the examination of the tumor tissue and the chronological examinations of the blood.

15. The method of analyzing a tumor according to claim 1, further comprising:
acquiring information concerning at least closed chromatin regions, in reference to the examination of the tumor tissue and the chronological examinations of the blood.

16. A system for analyzing a tumor, comprising:
an information processing apparatus that includes
a controller including an information acquiring section for acquiring information regarding an examination of a tumor tissue and chronological examinations of blood, an integrating section for integrating the information from the information acquiring section to generate data regarding the tumor, and an outputting section for outputting the data generated by the integrating section,
a storing section for storing the data output from the outputting section,
a displaying section for displaying the data output from the outputting section, and
a communicating section for presenting the data output from the outputting section onto a network;
a tumor tissue examination result outputting apparatus for outputting a result of the examination of the tumor tissue; and
a blood examination result outputting apparatus for outputting a result of the examinations of the blood.

17. A method of generating analyzed data of a tumor, comprising:
using an examination of a tumor tissue and chronological examinations of blood and having the examinations intervene in each other to integrally analyze data from the examinations, thereby generating data regarding the tumor.

18. The method of generating analyzed data of a tumor according to claim 17, further comprising:
acquiring mutational information regarding genes of the tumor tissue in reference to the examination of the tumor tissue; and
listing information concerning one or multiple genetic mutations in reference to the mutational information.

19. The method of generating analyzed data of a tumor according to claim 17, further comprising:
establishing one or multiple regions for the examination of the tumor tissue;
coupling biomarkers in an examination of blood to the regions; and
predicting states of the tumor in the regions by using the coupled biomarkers.

20. The method of generating analyzed data of a tumor according to claim 17, further comprising:
calculating a data set of clusters and features in reference to an information group regarding one or multiple biomarkers linked to tumor tissues and blood; and
estimating information regarding missing one or multiple biomarkers in reference to the data set.
